(19) United States
(12) Patent Application Publication
(10) Pub. No.: US 2005/0069904 A1
(43) Pub. Date: Mar. 31, 2005

(54) Analysis of real-time PCR data

(76) Inventors: Stuart Peirson, Seven Kings (GB); Jason Butler, London (GB)

Correspondence Address: MORGAN & FINNEGAN, L.L.P., 3 WORLD FINANCIAL CENTER, NEW YORK, NY, 10281-2101, US

(21) Appl. No.: 10/674,681

(22) Filed: Sep. 29, 2003

(51) Int. Cl. C12Q 1/68; G06F 19/00; G01N 33/48; G01N 33/50; C12P 19/34

(52) U.S. Cl. 435/6; 435/91.2; 702/20

(57) ABSTRACT

Data obtained from a polymerase chain reaction applied to a biological sample is analysed by putting the logarithmic dependence of fluorescence, or any other signal representative of the amount of reaction product, against cycle number. By fitting a straight line to that part of the curve that is substantially linear, both the intrinsic reaction efficiency and the initial loading of the reaction product may be determined.

### BACKGROUND OF THE INVENTION

The invention relates to the analysis of data obtained from a polymerase chain reaction (PCR) experiment.

Recent developments in PCR, together with new fluorescent techniques for detection of reaction products, have led to the introduction of real-time PCR, a technique now widely used in basic sciences, medical research and diagnostics. Real time PCR is preferred over other quantitative analysis methods, as it does not rely on the end-point of the reaction, which can be confounded by a variety of factors such as product inhibition, enzyme instability and a decrease in reactants as the reaction progresses. Quantitative analysis of real time PCR, on the other hand, is based on monitoring a fluorescence signal indicative of the amount of reaction product (“amplicon”) present after each cycle of PCR.

A PCR experiment, or assay, consist of a series of cycles of denaturing and annealing of the amplicon, theoretically resulting in a doubling of the amount of amplicon for each cycle. Practically, this ideal regime is never attained and the amount of amplicon grows with each cycle according to the exponential equation:
Xₙ=X₀·(1+E)ⁿ Equation 1
where Xₙ is the amount of amplicon after cycle n, X₀ is the amount of amplicon at the start and E is the efficiency of the reaction. If E=1, the amount of amplicon doubles every cycle (the theoretical limit) and if E=0, the reaction produces no additional amplicon.

The basic assumption underlying all analysis of PCR data is that the amount of amplicon in the reaction is proportional to a fluorescence signal from a fluorescent dye that becomes active when binding to double-stranded polynucleotides (e.g. SYBR (registered trademark of Molecular Probes, Inc.) Green I Mastermix by Applied Biosystems). Therefore, Equation 1 can be rewritten in terms of the fluorescence signal R:
Rₙ=R₀·(1+E)ⁿ Equation 2
where Rₙ is the fluorescence signal measured after cycle n and R₀ is the fluorescence signal corresponding to the starting concentration. FIG. 1 shows that, in a central region 1, the observed fluorescence signal does indeed show exponential behaviour in the number of cycles n.

Current analysis techniques of real-time PCR are based on estimating the initial concentration of a target amplicon in a sample relative to a control sample by determining the threshold cycles for the target amplicon in both the sample and the control, normalised by a reference amplicon that has the same starting concentration in both the sample and the control. The threshold cycle is the fractional cycle number at which a fixed amount of amplicon is formed. The difference between the sample and the control is the difference in the threshold cycle of the target and reference amplicons, which is then used to estimate how many fold the concentration of the target amplicon in the sample is larger (or smaller) than in the control.

A simple analysis might assure that the efficiency of the PCR is perfect, that is that the amount of reaction product doubles with every cycle of PCR. However, actual efficiencies tend to vary from the ideal value and the growth factor from one cycle to the next can be as low as 1.8, in practice. Due to the exponential growth underlying PCR, even small errors in the assumed efficiency can lead to extremely large errors in the estimated concentrations.

One recent approach, which does not assume perfect amplification efficiency, is described in Weihong Liu and David A. Saint, A New Quantitative Method of Real Time Reverse Transcription Polymerase Chain Reaction Assay Based On Simulation of Polymerase Chain Reaction Kinetics, Analytical Biochemistry 302, 52-589 (2002). As described with reference to FIG. 1 of this paper, Liu and Saint attempt to estimate the actual PCR efficiency in practice by selecting two points A and B on a fluorescent curve of the type shown in FIG. 1 of the present application, carefully choosing A and B such that the section of the curve between those two points is substantially a straight line by calculating the slope of that line, the actual efficiency can then be calculated. It is believed by the present applicant that this method will give substantially more accurate results than those which simply assume perfect efficiency, but probably less accurate results than the so called “standard curve” procedure, described below.

The standard curve approach requires the initial preparation of a series of standard samples established, for example by a dilution series, for the target and reference, to estimate the PCR efficiencies for the target and reference amplicons in the sample and in the control. Alternatively, on can assume that they are the same. However, this approach is time-consuming and labor intensive, given the need to create the standard curves. It further makes the (untested) assumption that reaction efficiency is not influenced by the dilution used to produce the standard curves.

It is an object of the present invention at least to alleviate these difficulties with the prior art. It is a further object, at least in some embodiments, to provide a convenient method of quantitative PCR analysis which can be automated, and in which results can be obtained in real time.

### SUMMARY OF THE INVENTION

In a first aspect of the invention, a method for analysing data from a polymerase chain reaction is provided, the reaction amplifying an amount of reaction product during a plurality of reaction cycles, including measuring a signal representative of the amount of reaction product for each of the cycles, and calculating a reaction by estimating a slope of the dependence of a logarithm of the signal on cycle number for a set of cycles over which the dependence is substantially linear.

In a second aspect of the invention, there is provided a method for calculating the efficiency of a polymerase chain reaction which runs for a plurality of cycles, from a dependence of a signal representative of an amount of reaction product on cycle number, wherein the efficiency is calculated by estimating a slope of the dependence of a logarithm of the signal on the cycle number for a set of cycles over which the dependence is substantially linear.

In a third aspect of the invention, a method for analysing data from a polymerase chain reaction is provided, the method including measuring a signal representative of an amount of reaction product for each of a plurality of cycles and analysing a dependence of the logarithm of the signal on the cycle numbers for a set of cycles over which the dependence is linear.

In a fourth aspect of the present invention, a system for analysing data from a polymerase chain reaction is provided, the reaction amplifying an amount of reaction product during a plurality of reaction cycles, the system including a memory for storing a signal representative of the amount of reaction product for each of the cycles, a processing unit for calculating a logarithm of the signal, a memory for storing the logarithm, and a reaction efficiency calculator, for calculating reaction efficiency from a dependence of the signal on the cycle number, the system further comprising a selector adapted to select a set of cycles over which the dependence of the logarithm on the cycle number is substantially linear, and wherein the efficiency calculator includes an estimator for estimating a slope of the dependence of the logarithm of the signal on the cycle number for the selected set of cycles.

In a fifth aspect of the present invention, a method for analysing data from polymerase chain reactions on a plurality of samples is provided, the reactions amplifying an amount of reaction product during a plurality of reaction cycles, including measuring a signal representative of the amount of reaction product for each of the cycles and each of the samples, calculating an average signal by averaging the signals obtained for each of the samples, and calculating a reaction efficiency by estimating a slope of the dependence of a logarithm of the averaged signal on the cycle number for a set of cycles over which the dependence is substantially linear.

In a sixth aspect of the present invention a method for analysing data from polymerase chain reactions applied to a plurality of samples, the reactions amplifying an amount of reaction product during a plurality of reaction cycles, including measuring a signal representative of the amount of reaction product for each of the cycles and each of the samples, and calculating a reaction efficiency by estimating a slope of the dependence of the logarithm of the averaged signal on the cycle number for a set of cycles over which the dependence is substantially linear, and calculating an average efficiency for the plurality of samples by averaging the efficiencies calculated for each of the samples.

In a seventh aspect of the present invention a medical diagnostic method is provided, the method comprising obtaining a biological sample, determining the efficiency of a polymerase chain reaction applied to the sample, the reaction amplifying an amount of reaction product during a plurality of reaction cycles, by measuring a signal representative of the amount of reaction product for each of the cycles, and estimating a slope of the dependence of a logarithm of the signal on cycle number for a set of cycles over which the dependence is substantially linear.

In a eighth aspect of the present invention, a method of calculating the initial load of a reaction product within a biological sample is provided, the method comprising applying a polymerase chain reaction to the sample over a plurality of cycles, the reaction amplifying the reaction product for each of the cycles, measuring a signal representative of the amount of reaction product for each of the cycles, and calculating the initial load by estimating a zero intercept of a line representative of a logarithm of the signal against cycle number for a set of cycles over which the dependence is substantially linear.

In an ninth aspect of the present invention, a medical diagnostic method is provided, the method comprising obtaining a biological sample, and applying a polymerase chain reaction to the sample over a plurality of cycles, the reaction amplifying the reaction product for each of the cycles, measuring a signal representative of the amount of reaction product for each of the cycles, and calculating the initial load by estimating a zero intercept of a line representative of a logarithm of the signal against cycle number for a set of cycles over which the dependence is substantially linear.

In a tenth aspect of the present invention, a method of genotyping is provided, the method comprising determining the gene level within a biological sample by creating a gene-based reaction product with the sample, applying a polymerase chain reaction to the sample over a plurality of cycles, the reaction amplifying the reaction product for each of the cycles, measuring a signal representative of the amount of reaction product for each of the cycles, and calculating the initial load by estimating a zero intercept of a line representative of a logarithm of the signal against cycle number for a set of cycles over which the dependence is substantially linear.

### BRIEF DESCRIPTION OF THE DRAWINGS

The invention may be carried into practice in a variety of ways and one specific embodiment will now be described, by way of example, with reference to the accompanying figures, in which:

FIG. 1 shows how the fluorescence varies with cycle number in a typical PCR experiment;

FIG. 2 shows a corresponding part of the log of the fluorescence, as used in the method of the preferred embodiment; and

FIG. 3 illustrates how reaction efficiency deteriorates as the number of cycles increases.

In the figures, like reference numerals refer to like features.

### DESCRIPTION OF THE PREFERRED EMBODIMENT

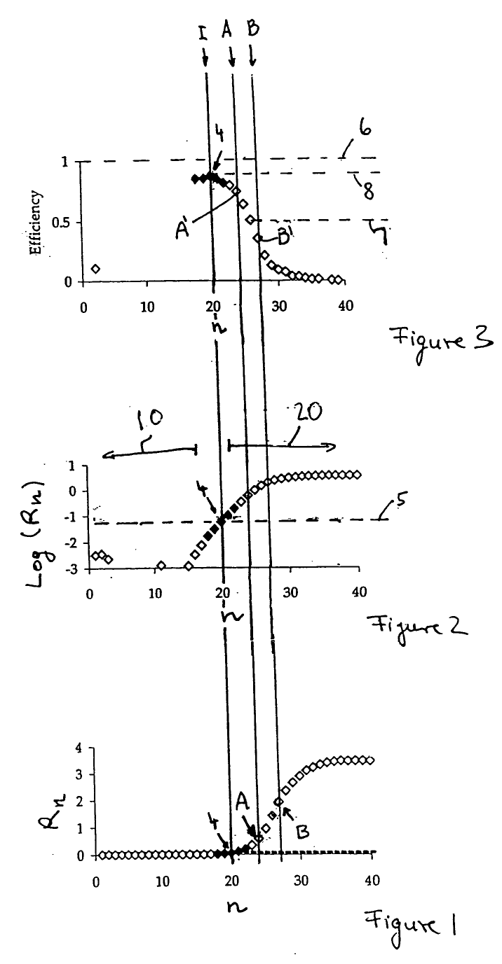

The present invention proceeds from the recognition, not previously known to have been noted by researchers in this field prior to the making of the present invention by the applicants, that significant further improvements in accuracy can be made by allowing for the fact that the PCR amplification efficiency is not in fact a constant at all. As shown in FIG. 3, if one plots the amplification efficiency against the cycle number, one sees that the efficiency rapidly drops as the reaction saturates. As may be seen from the figure, the actual efficiency never reaches the theoretical maximum, as shown by the dashed line 6 (an efficiency of 1 means that the amount of reaction product doubles with each cycle). However, note that the efficiency does not remain constant as has previously been assumed when performing quantitative PCR analysis. Indeed, by comparing FIGS. 1 and 3, it may be seen that Liu and Saint's points A and B (corresponding to points A prime and B prime in FIG. 3) effectively assume a fixed amplification efficiency somewhere between these points, as indicated by the dashed line 7. Although unknown by Liu and Saint, it now becomes clear that their calculations are based on a sub-optimal efficiency for the reaction as a whole. Note that they are (unknowingly) operating in a range within which the amplification efficiency is determined largely by saturation effects.

Once this point has been recognised, it now becomes clear that in order to avoid these saturation effects, one should be basing ones calculations on a range 4 of optimal efficiency, in other words at the intrinsic efficiency 8 of the reaction itself.

In the preferred embodiment of the present invention, this “intrinsic reaction” is determined by considering the logarithm of the curve of FIG. 1. Taking the logarithm (base 10) of Equation 2, a linear Equation in the number of cycles n is obtained:
log(Rₙ)=log(R₀)+n·log(1+E) Equation 3

The corresponding plot is shown at FIG. 2.

When linearly regressing the logarithm of the fluorescence signal at cycle n against cycle number n in the central region 1, that is fitting a linear equation of the form y=slope·x+intercept to the central region in which the log-linear plot in FIG. 2 is linear, R₀ and E can now be calculated directly from the coefficients of the regression. R₀, the fluorescence signal corresponding to the starting concentration, can be found from the regression as:
R₀=10^{intercept} Equation 4
and the efficiency E can be found similarly as:
E=10^{slope}−1 Equation 5

For this analysis to be valid, the linear regression should only be performed within a region for which the assumption of an exponential growth process is valid, or, equivalently, for which the log-linear plot of FIG. 2 is in fact a straight line. This region is illustrated in the figures by the reference numeral 4, and the filled-in data points. This central, linear region is bounded from below by a noisy region 10 where measurement noise dominates the fluorescence signal, which is therefore on average and to the limit of measurement accuracy, constant at the noise level Rₙₒᵢₛₑ. The linear region 4 is bounded from above by a saturation region 20, where the fluorescence signal approaches an asymptotic value R_{∞}, as the reaction saturates with increasing cycle number. For practical purpose, R_{∞} may in practice be approximated by the value of the fluorescence signal at the last cycle of the experiment and Rₙₒᵢₛₑ may be calculated as a multiple, for example by a factor of 1, of the standard deviation of the fluorescence signal calculated from the initial cycles, for example the first 10 cycles.

The number of samples within the set of cycles 4 to be analysed is selected by a user. Evidently, the set 4 has to contain at least two cycles, and the coefficient of determination of the fit can only be calculated for sets containing at least three cycles. Selecting the number of cycles represents a trade-off between minimising the effect of measurement noise by selecting as many cycles as possible while avoiding the selection of cycles which are in the boundary regions 10 and 20, for which the assumption of linearity is not valid. In practice, selecting four cycles was found to give good results: this corresponds to at least a 10-fold signal range included in the analysis (assuming minimal efficiency of 0.8).

If the set 4 contains an odd number of cycles, the set of cycles for use in the linear regression is selected by first finding a central cycle k (indicated in the drawings by the vertical line I) for which the logarithm of the fluorescence signal is closest to the average 5 log(R_{mid}) of the logarithm of the noise level log(Rₙₒᵢₛₑ) and the saturation level log(R_{∞}). A set of cycles is then selected which is centered on cycle k. If the set contains an even number of cycles, the set of cycles for use in the linear regression is selected by first finding two central cycles l and m for which the logarithm of the fluorescence signal is closest to the average log(R_{mid}). A set of cycles is then selected which is centered on cycles l and m. The average log(R_{mid}) is given by:
log(R_{mid})=(log(Rₙₒᵢₛₑ)+log(R_{∞}))/2 Equation 6
or, equivalently:
R_{mid}=(Rₙₒᵢₛₑ·R_{∞})^{0.5} Equation 7

It will be noted, turning back to FIG. 1, that the above analysis results in the so action of a sample set 4 which lies at the very bottom of the fluorescence curve, and well away from the points A and B used by Liu and Saint.

Once the set of cycles to be included in the linear regression is identified, linear regression is carried out as described above, and R₀ and E can be determined according to Equations 4 and 5. Thus, R₀ can be found directly from the linear regression analysis of the fluorescence data of a target amplicon and can then be used for further analysis, for example by normalising with respect to R₀ of a reference amplicon. Expression of the target amplicon in a sample, as compared to a control, for example, can then be assessed by comparing the normalised values of R₀ between the sample and the control. This kind of analysis requires that the concentration of the reference amplicon is the same in both the sample and the control.

Since most analytical approaches to analysing real-time PCR data involve the calculation of the threshold cycle C₁, the fractional cycle at which a fixed amount of amplicon has been produced, the efficiency calculated as set out above can be used to calculate R₀ from the threshold cycle:
R₀=R_{Ct}·(1+E)^{-Ct} Equation 8
where R_{Ct} is the fixed fluorescence threshold corresponding to the fixed amount of amplicon defining the threshold. This has the advantage that data commonly available from standard analysis packages can be used for the analysis. Furthermore, the efficiency can be used to normalise R_{0,T} of a target amplicon with respect to R_{0,R} of a reference amplicon directly from the threshold cycles, given that by definition R_{Ct,T}=R_{Ct,R}:
R_{0,T}/R_{0,R}=(1+E_{T})^{-Ct,T}/(1+E_{R})^{-Ct,R} Equation 9
where E_{R} and E_{T} are the efficiencies of the reference and target amplicons, respectively. The normalised R₀ for the sample and the control can than be compared to determine the relative expression of the target amplicon in a sample and in a control.

In order to test the accuracy of the method, dilution series were obtained for plasmid DNA and cDNA of the β-actin gene obtained from paired eyes obtained from wildtype mice. Paired whole eyes were homogenised in 0.5 ml of TriReagent (Sigma Aldrich) using Fastprep tubes in a FastPrep FP 120 (Q-Biogene). Total RNA was then extracted in TriReagent according to the manufacturer's instructions. RNA was resuspended at 60° C. in 20 μl of RNA Secure (Ambion). 1 μg of total RNA was then treated with 2 units of Rnase-Free Dnase (Sigma Aldrich) for thirty minutes at 37° C. to remove any traces of genomic DNA. Dnase-treated RNA was reverse transcribed with random decamers using a RetroScript kit (Ambion), according to the manufacturer's instructions. Once synthesised cDNA fidelity was tested by PCR, and samples were then stored at −20° C.

Primers for β-actin were designed using MacVector software (Accelrys, UK), and tested to ensure amplification of single discrete bands with no primer-dimers. Where possible, primers were designed to span introns to prevent genomic contamination. Primer sequences were as follows: Forward: 5′ACCAACTGGGACGATATGGAGAAGA 3′, β-actin reverse:5′cgcacgatttccctctcagc 3′ (403 bp product). All primers were synthesised by Sigma Genosys. PCR products were ligated into pGEM-T Easy vector (Promega) and transformed in DH5α competent cells (invitrogen). Minipreps of isolated plasmid DNA were then prepared (Promega). Before use, plasmid concentration was determined by spectrophotometry using an Eppendorf BioPhotometer and Serial dilutions were performed to give final concentrations between 10³-10⁶ copies. Dilution series of cDNA were composed of three tenfold dilutions of wildtype ocular cDNA. Real-time PCr was conducted using Sybr #Green I Mastermix (Applied Biosystems) using an ABI PRISM™ 7700 Sequence Detection System. Each reaction contained 1 μl of cDNA template along with 50 nM of primers in a final reaction volume of 25 μl. Cycling parameters were 95° C. for 10 minutes to activate DNA polymerase, then 40 cycles of 95° C. for 15 seconds, 60° C. for one minute with a final recording step of 78° C. for twenty seconds to prevent any primer-dimer formation. Melting curves were performed using Dissociation Curves software (Applied Biosystems) to ensure only a single product was amplified, and samples were also run on a 3% agarose gel to confirm specificity.

Table 1 shows data from β-actin dilution series analysed using R₀ values obtained, and shows a very close approximation to the actual dilutions used according to equation (R²>0.998). The results in table 1 clearly demonstrate the reliability of the method of the invention according to Equations 4 and 5 for estimating the efficiency and relative starting concentration of an amplicon in a PCR reaction, while avoiding the need to construct a standard curve by directly using the fluorescence signal for each sample analysed.

| Sample | Dilution | R₀ | Calculated dilution |
|---|---|---|---|
| B-actin plasmid | 1,000,000 copies | 4.063 × 10⁻⁷ | 1.000 |
| | 100,000 copies | 4.386 × 10⁻⁸ | 0.108 |
| | 10,000 copies | 3.299 × 10⁻⁹ | 0.008 |
| | 1,000 copies | 2.620 × 10⁻¹⁰ | 0.001 |
| B-actin cDNA | 1 | 8.805 × 10⁻⁸ | 1.000 |
| | 0.1 | 9.200 × 10⁻⁹ | 0.104 |
| | 0.01 | 8.618 × 10⁻¹⁰ | 0.010 |

If more than one sample is run, the method of the invention allows the differences in efficiency for the individual samples to be taken into account or, alternatively the efficiencies calculated from each of the sample can be averaged to provide an estimate of the underlying efficiency of the population of samples. The first option is advantageous if the efficiency varies significantly from one sample to the next, as there is then no implicit assumption that the efficiencies of the individual samples are equal. The second option of averaging individual efficiencies before calculating Ro is appropriate if the variability of the efficiency is relatively small and can be assumed to be due to measurement noise, rather than being due to differences in the “true” underlying efficiency. A decision between the two approaches can be based on an empirical cut-off for the standard deviation of the efficiencies. Alternatively the decision can be based or on the distribution of the efficiencies, for example by inspecting the histogram of efficiencies or using any appropriate clustering algorithm. For example, if the distribution of efficiencies were bimodal, the averaging of the whole population would not be appropriate as it is then likely that there are at least two underlying efficiencies. An alternative approach to pooling the experimental data, other than averaging the individual efficiencies, is to perform one single linear regression of log(Rₙ) against n for a complete data set including the data points from all samples. In the regime where the efficiencies are comparable across samples and, hence, pooling is appropriate, this will give very similar results to averaging the visual efficiencies.

By calculating an efficiency for each sample it is possible to apply statistical techniques to the analysis. For example, if the analysis involves a number of different types of samples from different sources, then a ANOVA may be used to determine if there are any statistically significant differences between samples from different sources, or if the observed variability is due to random noise. Furthermore, by using multiple measurements of the efficiency, a more reliable estimate of efficiency may be derived from the mean and confidence limits may be determined from the variance around the mean.

The invention finds applications in a number of different fields, including assay, investigating differences in gene expression, gene quantitation, genotyping, investigation of mutations, gene therapy, investigation of viral and bacterial loadings, and indeed any type of quantitative PCR analysis.

The description of the embodiment is not intended to limit the general applicability of the invention as a whole. For example, other signals than fluorescence obtained from fluorescent dye can be used as basis for the analysis, as long as the signal is representative of the amount of amplicon.

Estimation of the slope is not limited to linear regression, and simpler, model-free alternatives are possible. For example, the slope may be found by calculating the average of the difference between the signal measured for adjacent cycles of the selected set 4. Similarly, defining the cycles to be included in the set 4, can be achieved in alternative ways. For example, the number of cycles to be included in the analysis can be increased until a drop in the coefficient of determination of the associated linear regression is detected. The selected cycles do not necessarily have to be adjacent, nor necessarily centered on R_{mid}. Instead, the cycles in the set can be picked automatically and itteratively by determining the cycles to be included in the set such that the coefficient of determination of the linear regression is maximised.

Having described a particular preferred embodiment of the present invention, it is to be appreciated that the embodiment in question is exemplary only and that variations and modifications, such as will occur to those possessed of the appropriate knowledge and skills, may be made without departure from the spirit and scope of the invention as set forth in the appended claims.

1. A method for analysing data from a polymerase chain reaction, the reaction amplifying an amount of reaction product during a plurality of reaction cycles, including:
measuring a signal representative of the amount of reaction product for each of the cycles, and
calculating a reaction by estimating a slope of the dependence of a logarithm of the signal on cycle number for a set of cycles over which the dependence is substantially linear.

2. The method of claim 1, wherein the signal is a fluorescent signal.

3. The method of claim 1, wherein the slope is estimated from a linear regression.

4. The method of claim 1, wherein the slope is estimated by averaging the difference of the signal between the adjacent cycles over the set of cycles.

5. The method of claim 1, further including calculating a noise level and a saturation level for the signal and wherein,
if the set contains an odd number of cycles, the set of cycles is centered on the cycle for which the signal is closest to the average of the noise and saturation levels, and,
if the set contains an even number of cycles, the set of cycles is centered on the two cycles for which the signal is closest to the average of the noise and saturation levels.

6. The method of claim 5, wherein the slope is estimated from a linear regression and the number of points in the set is selected such that the coefficient of determination of the linear regression is maximised.

7. A method for calculating the efficiency of a polymerase chain reaction which runs for a plurality of cycles, from a dependence of a signal representative of an amount of reaction product on cycle number, wherein the efficiency is calculated by estimating a slope of the dependence of a logarithm of the signal on the cycle number for a set of cycles over which the dependence is substantially linear.

8. The method of claim 7, wherein the signal is a fluorescent signal.

9. The method of claim 7, wherein the slope is estimated from a linear regression.

10. The method of claim 7, wherein the slope is estimated by averaging the difference of the signal between adjacent cycles over the set of cycles.

11. The method of claim 7, including calculating a noise level and a saturation level for the signal and wherein,
if the set contains an odd number of cycles, the set of cycles is centered on the cycle for which the signal is closest to the average of the noise and saturation levels, and,
if the set contains an even number of cycles, the set of cycles is centered on the two cycles for which the signal is closest to the average of the noise and saturation levels.

12. The method of claim 11, wherein the slope is estimated from a linear regression and the number of points in the set is selected such that the coefficient of determination of the linear regression is maximised.

13. A method for analysing data from a polymerase chain reaction including measuring a signal representative of an amount of reaction product for each of a plurality of cycles and analysing a dependence of the logarithm of the signal on the cycle numbers for a set of cycles over which the dependence is linear.

14. The method of claim 13, including calculating a noise level and a saturation level for the signal; and wherein the set of cycles is centered on the cycle for which the signal is closest to the average of the noise and saturation levels.

15. A system for analysing data from a polymerase chain reaction, the reaction amplifying an amount of reaction product during a plurality of reaction cycles, the system including:
a memory for storing a signal representative of the amount of reaction product for each of the cycles,
a processing unit for calculating a logarithm of the signal,
a memory for storing the logarithm, and
a reaction efficiency calculator, for calculating reaction efficiency from a dependence of the signal on the cycle number;
the system further comprising a selector adapted to select a set of cycles over which the dependence of the logarithm on the cycles number is substantially linear, and
wherein the efficiency calculator includes an estimator for estimating a slope of the dependence of the logarithm of the signal on the cycle number for the selected set of cycles.

16. The system of claim 15, wherein the signal is a fluorescent signal.

17. The system of claim 15, wherein the estimator is arranged to linearly regress the signal against cycle number for the said set of cycles.

18. The system of claim 15, wherein the estimator is arranged to average the difference of the signal between adjacent cycles over the said set of cycles.

19. The system of claim 15, further including a processing unit for calculating a noise level and a saturation level for the signal and wherein the selector is arranged to select:
if the set contains an odd number of cycles, the set of cycles centered on the cycle for which the signal is closest to the average of the noise and saturation levels, and,
if the set contains an even number of cycles, the set of cycles centered on the two cycles for which the signal is closest to the average of the noise and saturation levels.

20. The system of claim 19, wherein the estimator is arranged to linearly regress the signal against cycle number for the said set of cycles.

21. The system of claim 20, wherein the selector is arranged to select the number of points in the set such that the coefficient of determination of the linear regression is maximised.

22. A method for analysing data from polymerase chain reactions on a plurality of samples, the reactions amplifying an amount of reaction product during a plurality of reaction cycles, including
measuring a signal representative of the amount of reaction product for each of the cycles and each of the samples,
calculating an average signal by averaging the signals obtained for each of the samples; and
calculating a reaction efficiency by estimating a slope of the dependence of a logarithm of the averaged signal on the cycle number for a set of cycles over which the dependence is substantially linear.

23. A method for analysing data from polymerase chain reactions applied to a plurality of samples, the reactions amplifying an amount of reaction product during a plurality of reaction cycles, including:
measuring a signal representative of the amount of reaction product for each of the cycles and each of the samples, and
calculating a reaction efficiency by estimating a slope of the dependence of the logarithm of the averaged signal on the cycle number for a set of cycles over which the dependence is substantially linear,
and calculating an average efficiency for the plurality of samples by averaging the efficiencies calculated for each of the samples.

24. The method of claim 1 in which the calculation is carried out in real-time as the polymerase chain reaction proceeds.

25. A medical diagnostic method comprising obtaining a biological sample, determining the efficiency of a polymerase chain reaction applied to the sample, the reaction amplifying an amount of reaction product during a plurality of reaction cycles, by measuring a signal representative of the amount of reaction product for each of the cycles, and estimating a slope of the dependence of a logarithm of the signal on cycle number for a set of cycles over which the dependence is substantially linear.

26. A method of calculating the initial load of a reaction product within a biological sample, comprising:
applying a polymerase chain reaction to the sample over a plurality of cycles, the reaction amplifying the reaction product for each of the cycles,
measuring a signal representative of the amount of reaction product for each of the cycles, and
calculating the initial load by estimating a zero intercept of a line representative of a logarithm of the signal against cycle number for a set of cycles over which the dependence is substantially linear.

27. A medical diagnostic method comprising obtaining a biological sample, and
applying a polymerase chain reaction to the sample over a plurality of cycles, the reaction amplifying the reaction product for each of the cycles,
measuring a signal representative of the amount of reaction product for each of the cycles, and
calculating the initial load by estimating a zero intercept of a line representative of a logarithm of the signal against cycle number for a set of cycles over which the dependence is substantially linear.

28. The method of claim 27 in which the reaction product is virus or bacteria based.

29. The method of claim 27 in which the reaction product is gene-based.

30. The method of claim 27 in which the reaction product comprises a vector-based product, the method being used to determine the amount of vector that has been taken up after gene therapy.

31. A method of genotyping comprising determining the gene level within a biological sample by:
creating a gene-based reaction product with the sample, applying a polymerase chain reaction to the sample over a plurality of cycles, the reaction amplifying the reaction product for each of the cycles,
measuring a signal representative of the amount of reaction product for each of the cycles, and
calculating the initial load by estimating a zero intercept of a line representative of a logarithm of the signal against cycle number for a set of cycles over which the dependence is substantially linear.

32. The method of claim 7 in which the calculation is carried out in real-time as the polymerase chain reaction proceeds.

33. The method of claim 13 in which the calculation is carried out in real-time as the polymerase chain reaction proceeds.

34. The method of claim 15 in which the calculation is carried out in real-time as the polymerase chain reaction proceeds.

35. The method of claim 22 in which the calculation is carried out in real-time as the polymerase chain reaction proceeds.


SEQUENCE LISTING (2 sequences)

SEQ ID NO: 1 - DNA, 25 bp, Mouse
  misc_feature: Forward b-Actin Primer
accaactgggacgatatggagaaga

SEQ ID NO: 2 - DNA, 20 bp, Mouse
  misc_feature: Reverse b-Actin primer
cgcacgatttccctctcagc